(19) United States
(12) Patent Application Publication
(10) Pub. No.: US 2009/0137749 A1
(43) Pub. Date: May 28, 2009

(54) PROCESSES FOR REDUCING ACID CONTENT OF A POLYALKYLENE TEREPHTHALATE AND USING SUCH IN THE PRODUCTION OF MACROCYCLIC POLYESTER OLIGOMER

(76) Inventors: Peter D. Phelps, Williamstown, MA (US); Paul M. Andrusyszyn, New Castle, DE (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/257,270

(22) Filed: Oct. 23, 2008

Related U.S. Application Data
(60) Provisional application No. 60/981,939, filed on Oct. 23, 2007.

(51) Int. Cl. C07C 69/76; C08L 67/03

(52) U.S. Cl. 525/437; 560/91

(57) ABSTRACT

The invention relates to a method for producing low-acid polyalkylene terephthalate from which MPO can be advantageously manufactured. In certain embodiments, the acid content of commercially available polybutylene terephthalate (PBT) is reduced by adding a small amount of 1,4-butane diol (BDO) to a solution of the commercially available PBT in refluxing ortho-dichlorobenzene (oDCB) solvent.

### RELATED APPLICATIONS

This application claims benefit of U.S. Provisional Patent Application No. 60/981,939, filed on Oct. 23, 2007, the text of which is incorporated herein by reference in its entirety.

### FIELD OF THE INVENTION

This invention relates generally to the manufacture of low-acid polyalkylene terephthalate (such as polybutylene terephthalate, PBT) for conversion to macrocyclic polyester oligomer (such as cyclic polybutylene terephthalate, cPBT). More particularly, in certain embodiments, the invention relates to the modification of commercially available polyalkylene terephthalate (such as PBT) with an alkane diol (such as 1,4-butane diol, BDO) as a pretreatment step to reduce acid content prior to conversion to macrocyclic polyester oligomer (such as cPBT).

### BACKGROUND OF THE INVENTION

International (PCT) Patent Application No. PCT/US2006/010541, (the '541 application) was filed on Mar. 24, 2006, was published as WO2006/104821 on May 10, 2006, and is incorporated herein by reference in its entirety. The '541 application describes systems and methods for preparing low-acid polyalkylene terephthalate (such as polybutylene terephthalate, PBT) from which macrocyclic polyester oligomer (MPO) can be advantageously manufactured. Depolymerization of low-acid polyalkylene terephthalate requires less catalyst and proceeds to equilibrium more quickly than depolymerization of higher-acid polyalkylene terephthalate. The use of less catalyst reduces the amount of residual oligomers formed, thereby reducing separation and filtration processing costs. The residual oligomer filtrate that does form is less gellular and easier to remove from a product stream when low catalyst concentrations are used.

In addition to its use in the production of MPO, the low-acid polyalkylene terephthalate is useful in its own right. For example, the low-acid polyalkylene terephthalate can be stabilized to prevent generation of acids, thereby resulting in reduced corrosion problems when used as a polymer in injection molding or other process applications.

In the '541 application, low-acid PBT is produced by reacting butanediol (BDO) and dimethylterephthalate (DMT) in an organic solvent such as ortho-dichlorobenzene (oDCB) in the presence of a catalyst at about atmospheric pressure and at about the boiling point of the solvent (for example, less than about 200° C.). However, low-acid PBT manufactured by this method may be more expensive than commercially-available PBT. Even though depolymerization of commercially-available PBT requires more catalyst and may result in an MPO product of lower quality, it may be less expensive to prepare MPO this way, depending on the relative scale of the manufacturing processes involved and the price of the feedstocks used.

There is a need for alternate methods of preparing low-acid polyalkylene terephthalate (such as polybutylene terephthalate, PBT) from which macrocyclic polyester oligomer (MPO) can be advantageously manufactured.

### SUMMARY OF THE INVENTION

The invention described in the present application relates to methods for producing low-acid polyalkylene terephthalate from which MPO can be advantageously manufactured. It is discovered that the acid content of commercially available polyalkylene terephthalates such as PBT can be reduced by adding a small amount of a diol such as 1,4-butane diol (BDO) to a solution of the commercially available polyalkylene terephthalate in a refluxing organic solvent, for example, ortho-dichlorobenzene (oDCB). The methods are preferably performed below about 240° C., and more preferably below about 200° C., and can be performed without a vacuum. The low-acid PBT can then be depolymerized to advantageously produce cyclic polybutylene terephthalate (cPBT).

Solid state polymerization methods use a nitrogen sweep to strip away BDO that is derived from transesterification reactions of diol-stopped polymer, thereby building low acid PBT at temperatures which inhibit additional acid formation. However, by adding BDO to commercially available (e.g., high-acid) PBT in refluxing organic solvent, it is discovered that low-acid PBT can be produced at temperatures below about 240° C. (preferably from about 170° C. to about 210° C.) without starting from dimethyl terephthalate (DMT) or terephthalic acid (TPA), and without performing solid state polymerization, as demonstrated by experiments described herein. Acid may be removed from feedstock PBT in a concentrated state (e.g., a PBT mixture within a range from about 30 wt. % to about 50 wt. % solids) by reaction with BDO under reflux, and then the PBT mixture is diluted (e.g., within a range from about 0.75 wt. % to about 1.5 wt. % solids, preferably at about 1 wt. % solids) for depolymerization, thereby producing MPO, for example, cyclic polybutylene terephthalate (cPBT).

In one aspect, the invention relates to a method for reducing the acid content of a commercially available polybutylene terephthalate (PBT) product, where the method includes the steps of: (a) maintaining a mixture at a temperature no greater than about 240° C. and a pressure at least about atmospheric pressure under solvent reflux, the mixture at least initially including a PBT product, 1,4-butanediol (BDO), an organic solvent, and a catalyst; and (b) removing water from the refluxing solvent. The solvent preferably includes ortho-dichlorobenzene. In certain embodiments, the mixture in step (a) is maintained at a polymer solids concentration within a range from about 30 wt. % to about 50 wt. %. In certain embodiments, the acid content of the PBT product is reduced from about 15 meq/kg or more to about 10 meq/kg or less. In certain embodiments, the acid content of the PBT product is reduced from about 30 meq/kg or more to about 10 meq/kg or less. In certain embodiments, the mixture in step (a) at least initially contains from about 2.0 g to about 15 g of 1,4-butanediol per kg of the PBT product. Preferably, the mixture in step (a) at least initially contains from about 3.7 to about 10.3 g of 1,4-butanediol per kg of the PBT product.

In another aspect, the invention relates to a method for preparing a macrocyclic polyester oligomer (MPO), the method including the steps of: (a) maintaining a mixture at a temperature no greater than about 240° C. and a pressure at least about atmospheric pressure under solvent reflux, removing water from the refluxing solvent, and maintaining a concentration of polymer solids in the mixture within a first range to produce a reduced-acid polyalkylene terephthalate product having acid content no greater than about 10 meq/kg, the mixture at least initially including a polyalkylene terephthalate product, a diol, an organic solvent, and a catalyst; and (b) reducing the concentration of polymer solids in the mixture following step (a) and maintaining the concentration of polymer solids in the mixture within a second range in the presence of heat, thereby depolymerizing the reduced-acid polyalkylene terephthalate product from step (a) to produce a MPO. The description of elements of the embodiments above can be applied to this aspect of the invention as well. In certain embodiments, the concentration of polymer solids in the mixture is maintained within a range from about 30 wt. % to about 50 wt. % in step (a), then reduced and maintained within a range from about 0.75 wt. % to about 1.5 wt. %. in step (b). The solvent preferably includes ortho-dichlorobenzene.

In certain embodiments, the polyalkylene terephthalate product includes butylene terephthalate units and/or ethylene terephthalate units. Step (a) is preferably conducted at a temperature between about 170° C. and about 210° C. In certain embodiments, the polyalkylene terephthalate product prior to step (a) has an acid content of at least about 15 meq/kg. In certain embodiments, step (b) further includes adding a depolymerization catalyst, which may or may not be the same as the catalyst in step (a). In certain embodiments, the mixture in step (b) includes a titanium depolymerization catalyst at a concentration no greater than about 2 mol Ti per 100 mol alkylene terephthalate repeat units. In certain embodiments, the mixture in step (b) includes a titanium depolymerization catalyst at a concentration from about 0.25 to about 1.25 mol Ti per 100 mol alkylene terephthalate repeat units.

In yet another aspect, the invention relates to a continuous or semi-continuous process for preparing a macrocyclic polyester oligomer by depolymerizing low-acid polybutylene terephthalate, the process including: (1) a first unit operation for reducing the acid content of a PBT product, wherein the first unit operation maintains a first mixture at a temperature no greater than about 240° C. and a pressure at least about atmospheric pressure under solvent reflux, the first mixture at least initially including PBT product, BDO, an organic solvent, and a catalyst, and wherein an output stream including the reduced-acid PBT product flows from the first unit operation to a second unit operation; and a second unit operation for depolymerization of the reduced-acid PBT, wherein the second unit operation exposes a second mixture including the reduced-acid PBT to heat in the presence of a depolymerization catalyst, thereby producing a macrocyclic polyester oligomer. In certain embodiments, the first mixture at least initially contains from about 2.0 g to about 15 g of 1,4-butanediol per kg of the PBT product. Preferably, the first mixture at least initially contains from about 3.7 to about 10.3 g of 1,4-butanediol per kg of the PBT product.

In preferred embodiments, water is removed from refluxing solvent in the first unit operation. In certain embodiments, the concentration of polymer solids in the first mixture is maintained within a range from about 30 wt. % to about 50 wt. %, and the polymer solids concentration of the second mixture is maintained within a range from about 0.75 wt. % to about 1.5 wt. %. The solvent preferably includes ortho-dichlorobenzene.

In certain embodiments, the first unit operation reduces acid content of the PBT product from about 15 meq/kg or more to about 10 meq/kg or less. In certain embodiments, the first unit operation reduces acid content of the PBT product from about 30 meq/kg or more to about 10 meq/kg or less.

In certain embodiments, the second mixture includes a titanium depolymerization catalyst at a concentration no more than about 2 mol Ti per 100 mol butylene terephthalate repeat units. In certain embodiments, the second mixture includes a titanium depolymerization catalyst at a concentration from about 0.25 to about 1.25 mol Ti per 100 mol butylene terephthalate repeat units.

The description of the embodiments of one aspect of this invention may be applied to other aspects of the invention as well.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 is a process flow diagram depicting unit operations in a process for producing a low-acid polyalkylene terephthalate, according to an illustrative embodiment of the invention.

FIG. 2 is a process flow diagram depicting unit operations in a process for producing a macrocyclic polyester oligomer by polymerizing and subsequently depolymerizing/cyclizing a low-acid polyalkylene terephthalate, according to an illustrative embodiment of the invention.

FIG. 3 is a graph depicting PBT molecular weight as a function of reaction time in a reaction mixture according to an illustrative embodiment of the invention.

### DETAILED DESCRIPTION

It is contemplated that compositions, mixtures, systems, methods, and processes of the claimed invention encompass variations and adaptations developed using information from the embodiments described herein. Adaptation and/or modification of the compositions, mixtures, systems, methods, and processes described herein may be performed by those of ordinary skill in the relevant art.

Throughout the description, where systems are described as having, including, or comprising specific components, or where processes and methods are described as having, including, or comprising specific steps, it is contemplated that, additionally, there are systems of the present invention that consist essentially of, or consist of, the recited components, and that there are processes and methods according to the present invention that consist essentially of, or consist of, the recited processing steps.

Similarly, where mixtures and compositions are described as having, including, or comprising specific compounds and/or materials, it is contemplated that, additionally, there are mixtures and compositions of the present invention that consist essentially of, or consist of, the recited compounds and/or materials.

It should be understood that the order of steps or order for performing certain actions is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

The '541 application, describes systems and methods for preparing low-acid polyalkylene terephthalate from which macrocyclic polyester oligomer (MPO) can be advantageously manufactured, for example, using less titanium catalyst. The invention described in the present application introduces an alternate approach for producing low-acid polyalkylene terephthalate from which MPO can be advantageously manufactured. In certain embodiments, the acid content of commercially available polybutylene terephthalate (PBT) is reduced by adding a small amount of 1,4-butane diol (BDO) to a solution of the commercially available PBT in refluxing ortho-dichlorobenzene (oDCB) solvent. The low-acid PBT is then advantageously depolymerized to form cyclic polybutylene terephthalate (cPBT).

In other embodiments, a low-acid PBT is formed by reacting a diol-rich pre-polymer with low molecular weight, acid-stopped linears in refluxing ortho-dichlorobenzene (oDCB) solvent, such linears being a by-product of an MPO production process (e.g., a by-product of the depolymerization of PBT to form cPBT). The low-acid PBT is then advantageously depolymerized to form cPBT.

Low-acid PBT may also be prepared using a combination of the methods presented, e.g., preparation from (i) commercially-available PBT and BDO in oDCB, (ii) reaction of linear oligomer recyclate and diol-rich PBT pre-polymer in oDCB, and/or (iii) reaction of BDO and DMT in oDCB. The low-acid PBT and may then be depolymerized to form cPBT, according to methods described herein.

Low-acid PBT prepared as described herein may also be isolated and stabilized to prevent acid formation, and used as an engineering thermoplastic resin. Such low-acid PBT resins exhibit improved polymer properties, for example, increased hydrolytic and thermal stability, due to the low-acid content.

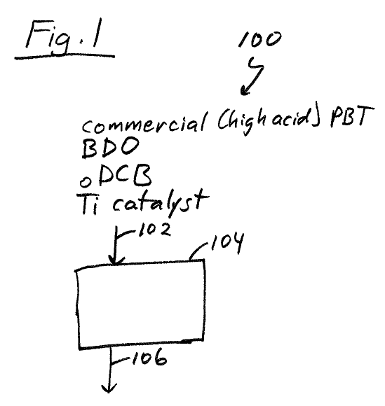

FIG. 1 is a flow diagram 100 depicting a process for producing a low-acid polyalkylene terephthalate, according to an illustrative embodiment of the invention. In the example depicted in FIG. 1, commercially-available (high acid) PBT reacts with a small amount of BDO in oDCB solvent in the presence of a catalyst to produce low acid PBT. One or more input streams 102 provide reactants including commercially-available PBT and BDO in a reactor 104. The one or more input streams 102 also provide solvent (e.g., ortho-dichlorobenzene, oDCB), and a titanium catalyst (e.g., tetraisopropyl titanate, TPT). The reaction mixture is maintained at about the boiling point of the solvent at atmospheric pressure, and water is removed from the refluxing solvent. The output stream 106 may be filtered for removal of non-PBT species, and/or stabilized to prevent formation of acid species. The low-acid PBT filtered from the output stream 106 may be pelletized, shaped, or otherwise processed so that the resulting PBT product is in a form that is convenient for use or transport. Alternatively, the low-acid PBT may be used directly as input in a depolymerization/cyclization process for the advantageous production of cPBT, as described elsewhere herein.

Typical commercial grades of PBT (feedstock PBT) have degree of polymerization from about 80 to about 220. It is desired to use enough BDO to convert substantially all of the acid end groups of the feedstock PBT to alcohols (hydroxybutyl ester end groups). It is found that use of BDO in an amount from about 2.0 g to about 15 g BDO per kg of PBT provides sufficient conversion of acid end groups, and, preferably, from about 3.7 to about 10.3 g BDO per kg of PBT.

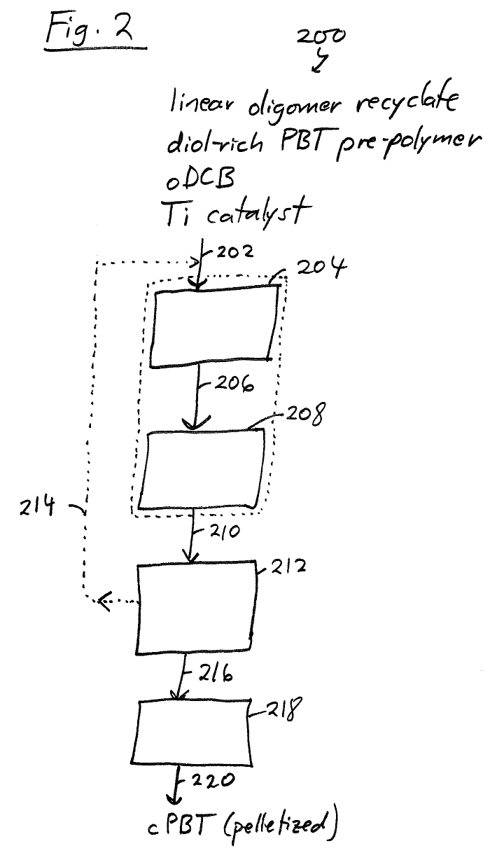

FIG. 2 is a flow diagram 200 depicting a process for producing a macrocyclic polyester oligomer by preparing a low-acid polyalkylene terephthalate (e.g., low-acid PBT) and subsequently depolymerizing/cyclizing the low-acid polyalkylene terephthalate to form MPO (e.g., cPBT). In the example depicted in FIG. 2, linear oligomer recyclate and diol-rich PBT pre-polymer react to produce low-acid PBT, which is depolymerized (cyclized) to form cPBT. The output stream 206 of the reaction step 204 contains low-acid PBT and is used as input in a depolymerization (cyclization) step 208. It may not be necessary to transfer the polymerization output 206 from one vessel to another, because depolymerization may be conducted using one or more of the reaction vessel(s) used in the reaction step 204. In one embodiment, a single unit operation includes both the production of low-acid polyalkylene terephthalate and depolymerization steps. In certain embodiments, the low-acid PBT is, essentially, an intermediate in the production of cPBT. The low-acid PBT produced thusly may be allowed to build to a molecular weight determined to provide improved overall cPBT production rate and/or properties.

The unit operations depicted in the figures may include input and output streams in addition to those shown. For example, in FIG. 2, solvent may be added to dilute the product of the reaction step 204 to levels required for the depolymerization step 208. The process streams shown may contain components other than those listed. The representative contents of process streams are provided for convenience.

In the process of FIG. 2, an output stream 210 of the depolymerization reaction may contain cPBT product in oDCB solvent, as well as byproducts including, for example, residual oligomer, catalyst residue, THF complexes, non-MPO macrocyclic material, and other compounds. The depolymerization output stream 210 may undergo filtration and/or other separation processing 212 so that cPBT product 216 and/or residual oligomers 214 may be extracted. The residual oligomers 214 may be recycled and used as part or all of the linear oligomer recyclate in the input stream 202. The cPBT product 216 can undergo pelletization and/or shaping 218 for conversion into an easily-transportable form 220.

A recyclate stream 214 rich in residual oligomer including, for example, carboxylic acid-terminated linear oligomer species, may be separated from the depolymerization output stream 210. The residual oligomer-rich stream 214 can then be used as input in the reaction step 204, thereby increasing overall conversion of monomers to cPBT. It is preferable, but not required, to remove catalyst residue before using oligomer byproduct as recyclate in the reaction step 204, for example, using methods described in the '541 application, incorporated by reference herein.

### EXPERIMENTAL EXAMPLES

Experimental examples described herein demonstrate the synthesis of low-acid PBT from: (i) commercially-available PBT and BDO in oDCB, and (ii) reaction of linear oligomer recyclate and diol-rich PBT pre-polymer in oDCB. The low-acid PBT formed thereby was then depolymerized to form cPBT.

Experiments 1 and 2 are control experiments in which no BDO was added. In Experiments 3-10, BDO was added to oDCB solutions of commercially-available PBT pellets at a concentration of about 40 wt. % solids. The commercially-available PBT that was used included Valox 315 grade PBT from GE Plastics and 6550 grade PBT from BASF. The Valox resin had a starting acid concentration of 39.2 mmol/kg and the BASF 6650 resin had a starting acid concentration of 23.7 mmol/kg. In each of Experiments 1-10, a IL 3-necked round bottom flask equipped with a mechanical stirrer, a heated reflux condenser fitted with a short path distillation head with receiver, and an inert gas inlet was charged with PBT (220 g or 1 mol repeat units), BDO (various amounts ranging from 235-1140 mg or 0.26 to 1.26 mol % of repeat units), anhydrous oDCB solvent (330 g per 220 g PBT to give 40% reactant solids) and 35 mg fresh of TPT catalyst. BDO and oDCB were used without further purification. The reactants were heated to reflux at about 187° C. (at 40% solids the atmospheric boiling point is about 187° C.) and the reacting mixture was sampled to determine the effect on molecular weight (Mw) as equilibration of added BDO took place. After the reaction was held at reflux for the desired time, the polymer solution was poured into a jar. Samples of the 40% PBT/oDCB solid were dried in vacuo at 100° C. to yield dried, powdered polymer.

The PBT powder was then depolymerized to form cPBT. A flame dried, 3-necked 250 ml round bottom flask equipped with a mechanical stirrer, a short path distillation head and condenser, and an inert gas inlet was charged with PBT (approximately 7 mmol or 1.54 g dry wt pellets or powder from solution polymerization to nearest 0.1 mg) and anhydrous oDCB (approximately 10 ml or 143 g to nearest 0.1 mg), then submerged into 220 C oil bath. After the PBT dissolved and several mL of solvent distilled over head, to insure dryness of the reaction, 50 ul of freshly prepared catalyst solution was added to provide an initial Ti concentration of 0.7 mol % vs. PBT repeat units. The catalyst solution was Ti(BD:HG) (4:1) at concentration of 1M in Ti and was prepared according to the methods described in the '541 application, incorporated herein by reference. The reaction was then maintained under a positive pressure of dry nitrogen and sampled at 5, 10 and 15 minutes to determine the initial rate of CBT formation. For the indicated experiments, additional catalyst was added at 15 minutes and a final sample of the reaction was taken at 2 hrs to determine extent of CBT formation by HPLC technique.

In Experiment 11, low-acid PBT was prepared by reacting linear oligomer recyclate and diol-rich PBT pre-polymer in oDCB. Low molecular weight, diol-stopped PBT pre-polymer was prepared by the solution polymerization procedure described in the '541 application, incorporated herein by reference, using 4% excess BDO. DMT and BDO were reacted in oDCB at atmospheric pressure at about the boiling point of oDCB in the presence of TPT to prepare the PBT pre-polymer. The PBT pre-polymer had a molecular weight of 20.2K and had acid concentration less than 1 mmol/kg, such that virtually all end groups were alcohols. The linear oligomer recyclate consisted essentially of titanium-free (filtered) acid-stopped linears, isolated from a byproduct of PBT depolymerization (cPBT production). Filtration at 180° C. was performed using dried linear waste cake from a cPBT production facility, as described in the above-referenced international patent application. The acid-stopped linears contained 238 mmol/kg acid and were virtually free of titanium.

A 1 L 3-necked round bottom flask equipped with a mechanical stirrer, a heated reflux condenser, and an inert gas inlet was charged with solids (75 wt. % diol-capped PBT pre-polymer and 25 wt. % Ti-free linears), anhydrous oDCB solvent (330 g per 220 g solids to give 40% reactant solids), and 35 mg fresh of TPT catalyst (initial Ti concentration of 0.7 mol % vs. PBT repeat units). oDCB was used without further purification. The reactants were heated to reflux at about 187° C. (at 40% solids the atmospheric boiling point is about 187 C). After the reaction was held at reflux for seven hours, the polymer solution was poured into a jar. Samples of the 40% PBT/oDCB solid were dried in vacuo at 100° C. to yield dried, powdered polymer.

The PBT powder was then depolymerized to form cPBT. A flame dried, 3-necked 250 ml round bottom flask equipped with a mechanical stirrer, a short path distillation head and condenser, and an inert gas inlet was charged with PBT (approximately 7 mmol or 1.54 g dry wt pellets or powder from solution polymerization to nearest 0.1 mg) and anhydrous oDCB (approximately 110 ml or 143 g to nearest 0.1 mg), then submerged into 220 C oil bath. After the PBT dissolved and several mL of solvent distilled over head, to insure dryness of the reaction, 50 ul of freshly prepared catalyst solution was added to provide an initial Ti concentration of 0.7 mol % vs. PBT repeat units. The catalyst solution was Ti(BD:HG) (4:1) at concentration of 1M in Ti and was prepared according to the methods described in the above-referenced international patent application. The reaction was then maintained under a positive pressure of dry nitrogen and sampled at 5, 10 and 15 minutes to determine the initial rate of CBT formation. Additional catalyst was added at 15 minutes and a final sample of the reaction was taken at 2 hrs to determine extent of CBT formation by HPLC technique.

Table 1 shows data obtained from certain of Experimental Examples #3-10 demonstrating the reduction of the acid content of commercially-available PBT by addition of BDO to refluxing solution.

TABLE 1
Preparation of Low-acid PBT from Commercially-available PBT

| PBT Type (Expt. #) | BDO/220 g PBT | | Reflux Time | | Initial Acid in PBT | Final Acid | | Mw vs PS |
|---|---|---|---|---|---|---|---|---|
| BASF 6550 (#3) | 470 | mg | 23 | hr | 23.7 mmol/Kg | 8.6 | mmol/Kg | 76.3K |
| BASF 6550 (#5) | 470 | mg | 1 | hr | 23.7 mmol/Kg | 8.6 | mmol/Kg | 55K |
| BASF 6550 (#6) | 940 | mg | 7 | hr | 23.7 mmol/Kg | 6.8 | mmol/Kg | 55.2K |
| Valox 315 (#8) | 235 | mg | 23 | hr | 39.2 mmol/Kg | 28.1 | mmol/Kg | 80.7K |
| Valox 315 (#9) | 940 | mg | 1 | hr | 39.2 mmol/Kg | 11.0 | mmol/Kg | 56.0K |
| Valox 315 (#10) | 1175 | mg | 2 | hr | 39.2 mmol/Kg | 8.4 | mmol/Kg | 56.0K |

Table 1 demonstrates the effect of the reaction time and the amounts of BDO added on the acid content and final molecular weight of the PBT resin. The acid content of commercially-available PBT was significantly reduced by the methods described above (e.g., from 23.7 mmol/kg to less than 10 mmol/Kg, and from 39.2 mmol/Kg to less than 30, 25, 20, 15, or 10 mmol/Kg).

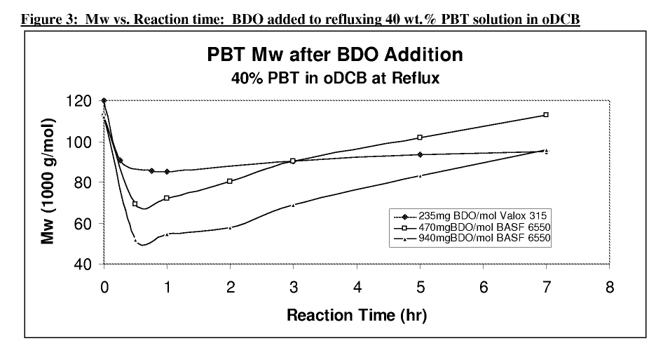

Several of the equilibration reactions were monitored for molecular weight over the course of the reaction. FIG. 3 is a chart showing how molecular weight drop is related to the level of BDO added and how molecular weight recovers over time as end groups react with each other. Using more BDO results in a lower-acid PBT, but causes a steeper molecular weight drop-off. Thus, the amount of BDO and the reaction time can be chosen for the desired acid reduction and molecular weight of the PBT to be depolymerized to form MPO.

Table 2 shows data obtained for Experimental Examples #1-11 during depolymerization of low-acid PBT. The low-acid PBT for experiments #1-11 was prepared by the various methods described above, as denoted in Table 2 by the indicated amounts of initial reactants (e.g., commercial PBT, oDCB, BDO, diol-capped PBT, and/or Ti-free linears) and the indicated solution polycondensation (SP) time (e.g., 1, 2, 7, or 23 hours). Table 2 shows cPBT concentration as a function of time, along with the initial cPBT production rate, and the acid concentration and molecular weight of the PBT after SP, just prior to depolymerization.

TABLE 2
Depolymerization of Low-acid PBT to form cPBT

| Depolymerization to form cPBT | | | cPBT Initial | Acid conc. of | |
|---|---|---|---|---|---|
| (180 C. Depoly at 0.07M in oDCB | Time | cPBT | Rate | PBT after SP | Mw PBT |
| 0.7% Catalyst) | (hr) | g/L | (g/L/hr) | (mmol/Kg) | (1000 g/mol) |
| Example #1 (control) | 0.083 | 0.273 | 0.58 | 39.2 | 71 |
| Valox 315 + trace TPT | 0.167 | 0.326 | | | |
| No added BDO | 0.250 | 0.370 | | | |
| 0.7% then 3% Ti at 15 min | 2.000 | 9.137 | | | |
| Example #2 (control) | 0.083 | 0.558 | 6.17 | 23.7 | 114 |
| BASF 6550 depoly | 0.167 | 1.117 | | | |
| No added BDO | 0.250 | 1.586 | | | |
| 0.7% Ti cat at t = 0 | 2.000 | 5.559 | | | |
| no additional cat at 15 min | | | | | |
| Example #3 | 0.083 | 1.126 | 10.2 | 8.6 | 76.3 |
| 220 g 6550 40% in oDCB | 0.167 | 1.970 | | | |
| 470 mg BDO 23 hr SP | 0.250 | 2.823 | | | |
| 0.7% then 3% Ti at 15 min | 2.000 | 9.207 | | | |
| Example #4 | 0.083 | 1.419 | 10.6 | 6.8 | 65.6 |
| 220 g 6550 40% in oDCB | 0.167 | 2.341 | | | |
| 940 mg BDO 7 hr SP | 0.250 | 3.192 | | | |
| 0.7% then 3% Ti at 15 min | 2.000 | 8.799 | | | |
| Example #5 | 0.083 | 0.930 | 9.27 | 8.6 | 55 |
| 220 g 6550 40% in oDCB | 0.167 | 1.762 | | | |
| 470 mg BDO 1 hr SP | 0.250 | 2.475 | | | |
| no additonal cat at 15 min | 2.000 | 7.761 | | | |
| Example #6 | 0.083 | 1.283 | 11.5 | 6.8 | 55.2 |
| 220 g 6550 40% in oDCB | 0.167 | 2.250 | | | |
| 940 mg BDO 7 hr SP | 0.250 | 3.191 | | | |
| no additonal cat at 15 min | 2.000 | 8.447 | | | |
| Example #7 | 0.083 | 0.150 | 1.07 | 39.8 | 120 |
| Valox 315 depoly | 0.167 | 0.234 | | | |
| 0.7% Ti cat at t = 0 | 0.250 | 0.329 | | | |
| no additonal cat at 15 min | 2.00 | 1.460 | | | |
| Example #8 | 0.083 | 0.208 | 1.48 | 28.1 | 80.7 |
| 220 g Valox 315 40% in oDCB | 0.167 | 0.292 | | | |
| 235 mg BDO 23 hr SP | 0.250 | 0.455 | | | |
| 0.7% then 3% Ti at 15 min | 2.000 | 9.074 | | | |
| Example #9 | 0.083 | 0.670 | 3.80 | 11 | 56.0 |
| 220 g Valox 315 40% in oDCB | 0.167 | 1.048 | | | |
| 940 mg BDO 1 hr SP | 0.250 | 1.303 | | | |
| no additonal cat at 15 min | 2.000 | 3.776 | | | |
| Example #10 | 0.083 | 1.424 | 9.82 | 8.4 | 56.0 |
| 220 g Valox 315 40% in oDCB | 0.167 | 2.349 | | | |
| 1175 mg BDO 2 hr SP | 0.250 | 3.061 | | | |
| no additonal cat at 15 min | 2.000 | 8.401 | | | |
| Example #11 | 0.083 | 1.302 | 10.9 | 5.8 | 58.5 |
| 75% 20K diol capped | 0.167 | 2.290 | | | |
| 25% Ti Free linears 7 hr SP | 0.250 | 3.118 | | | |
| 0.7% then 3% Ti at 15 min | 2.000 | 8.610 | | | |

Table 2 shows that the acid level in the starting PBT was significantly reduced by both the addition of BDO to commercially-available PBT in refluxing oDCB solvent (Example #3-10), as well as by the reaction of diol-capped PBT pre-polymer with Ti-free linear recyclate in refluxing oDCB solvent (Example #11). Table 2 also shows favorable rates of cPBT formation and ultimate cPBT conversion using only 0.7 mol % Ti catalyst when a low-acid PBT is used. In some cases, the initial rate of conversion to cPBT is significantly increased using the low-acid PBT versus commercially-available PBT (e.g., rates of conversion of at least about 7, 8, 9, 10, or 11 g/L/hr), leading to more efficient, less costly MPO production. Table 2 also shows that it is possible to forego use of additional catalyst (e.g., after 15 minutes of depolymerization), and still obtain high ultimate cPBT conversion rates (e.g., see Example #6). Other processing advantages of using a low-acid PBT for depolymerization to cPBT are as described in the above-referenced '541 application.

### EQUIVALENTS

While the invention has been particularly shown and described with reference to specific preferred embodiments, it should be understood by those skilled in the art that various changes in form and detail may be made therein without departing from the spirit and scope of the invention as defined by the appended claims.

What is claimed is:

1. A method for reducing the acid content of a commercially available polybutylene terephthalate product, the method comprising the steps of:
(a) maintaining a mixture at a temperature no greater than about 240° C. and a pressure at least about atmospheric pressure under solvent reflux, the mixture at least initially comprising:
(i) a polybutylene terephthalate product;
(ii) 1,4-butanediol;
(iii) an organic solvent; and
(iv) a catalyst; and
(b) removing water from the refluxing solvent.

2. The method of claim 1, wherein the solvent comprises ortho-dichlorobenzene.

3. The method of claim 1, wherein the mixture in step (a) is maintained at a polymer solids concentration within a range from about 30 wt. % to about 50 wt. %.

4. The method of claim 1, wherein acid content of the polybutylene terephthalate product is reduced from about 15 meq/kg or more to about 10 meq/kg or less.

5. The method of claim 1, wherein acid content of the polybutylene terephthalate product is reduced from about 30 meq/kg or more to about 10 meq/kg or less.

6. A method for preparing a macrocyclic polyester oligomer, the method comprising the steps of:
(a) maintaining a mixture at a temperature no greater than about 240° C. and a pressure at least about atmospheric pressure under solvent reflux, removing water from the refluxing solvent, and maintaining a concentration of polymer solids in the mixture within a first range to produce a reduced-acid polyalkylene terephthalate product having acid content no greater than about 10 meq/kg, the mixture at least initially comprising:
(i) a polyalkylene terephthalate product;
(ii) a diol;
(iii) an organic solvent; and
(iv) a catalyst; and
(b) reducing the concentration of polymer solids in the mixture following step (a) and maintaining the concentration of polymer solids in the mixture within a second range in the presence of heat, thereby depolymerizing the reduced-acid polyalkylene terephthalate product from step (a) to produce a macrocyclic polyester oligomer.

7. The method of claim 6, wherein step (a) comprises maintaining a concentration of polymer solids in the mixture within a first range from about 30 wt. % to about 50 wt. %, and step (b) comprises maintaining a concentration of polymer solids in the mixture within a range from about 0.75 wt. % to about 1.5 wt. %.

8. The method of claim 6, wherein the solvent comprises ortho-dichlorobenzene.

9. The method of claim 6, wherein the polyalkylene terephthalate product comprises butylene terephthalate units and/or ethylene terephthalate units.

10. The method of claim 6, wherein step (a) is conducted at a temperature between about 170° C. and about 210° C.

11. The method of claim 6, wherein the polyalkylene terephthalate product prior to step (a) has at least about 15 meq/kg acid content.

12. The method of claim 6, wherein step (b) further comprises adding a depolymerization catalyst.

13. The method of claim 6, wherein the mixture in step (b) comprises a titanium depolymerization catalyst at a concentration no greater than about 2 mol Ti per 100 mol alkylene terephthalate repeat units.

14. The method of claim 6, wherein the mixture in step (b) comprises a titanium depolymerization catalyst at a concentration from about 0.25 mol Ti per 100 mol alkylene terephthalate repeat units to about 1.25 mol Ti per 100 mol alkylene terephthalate repeat units.

15. A continuous or semi-continuous process for preparing a macrocyclic polyester oligomer by depolymerizing low-acid polybutylene terephthalate, the process comprising:
a first unit operation for reducing the acid content of a polybutylene terephthalate product, wherein the first unit operation maintains a first mixture at a temperature no greater than about 240° C. and a pressure at least about atmospheric pressure under solvent reflux, the first mixture at least initially comprising the polybutylene terephthalate product, 1,4-butanediol, an organic solvent, and a catalyst, and wherein an output stream comprising the reduced-acid polybutylene terephthalate product flows from the first unit operation to a second unit operation; and
a second unit operation for depolymerization of the reduced-acid polybutylene terephthalate, wherein the second unit operation exposes a second mixture comprising the reduced-acid polybutylene terephthalate to heat in the presence of a depolymerization catalyst, thereby producing a macrocyclic polyester oligomer.

16. The process of claim 15, wherein the polymer solids concentration of the first mixture is maintained within a first range from about 30 wt. % to about 50 wt. %, and wherein the polymer solids concentration of the second mixture is maintained within a range from about 0.75 wt. % to about 1.5 wt. %.

17. The process of claim 15, wherein the solvent comprises ortho-dichlorobenzene.

18. The process of claim 15, wherein the first unit operation reduces acid content of the polybutylene terephthalate product from about 15 meq/kg or more to about 10 meq/kg or less.

19. The process of claim 15, wherein the first unit operation reduces acid content of the polybutylene terephthalate product from about 30 meq/kg or more to about 10 meq/kg or less.

20. The process of claim 15, wherein the second mixture comprises a titanium depolymerization catalyst at a concentration no greater than about 2 mol Ti per 100 mol butylene terephthalate repeat units.

21. The process of claim 15, wherein the second mixture comprises a titanium depolymerization catalyst at a concentration from about 0.25 mol Ti per 100 mol butylene terephthalate repeat units to about 1.25 mol Ti per 100 mol butylene terephthalate repeat units.

22. The process of claim 15, wherein water is removed from refluxing solvent in the first unit operation.

23. The method of claim 1, wherein the mixture in step (a) at least initially contains from about 2.0 to about 15 g of 1,4-butanediol per kg of polybutylene terephthalate.

24. The method of claim 1, wherein the mixture in step (a) at least initially contains from about 3.7 to about 10.3 g of 1,4-butanediol per kg of polybutylene terephthalate.

25. The process of claim 15, wherein the first mixture at least initially contains from about 2.0 to about 15 g of 1,4-butanediol per kg of polybutylene terephthalate.

26. The process of claim 15, wherein the first mixture at least initially contains from about 3.7 to about 10.3 g of 1,4-butanediol per kg of polybutylene terephthalate.
